SEQUENCE LISTING (42 sequences)

SEQ ID NO: 1 - protein, 23 aa, Artificial Sequence
  synthetic sequence
RGKRGGHATNYRIVAPRSRDERG

SEQ ID NO: 2 - protein, 23 aa, Artificial Sequence
  synthetic sequence
ESLSAIEAELEKVAHQLQALRRG

SEQ ID NO: 3 - protein, 23 aa, Artificial Sequence
  synthetic sequence
KISGGPHISYPPLHEWVLREGEE

SEQ ID NO: 4 - protein, 23 aa, Artificial Sequence
  synthetic sequence
KISGGPHISYPPLHEWVLREGAA

SEQ ID NO: 5 - protein, 24 aa, Artificial Sequence
  synthetic sequence
LRELDLAQEKTGWFALGSRSHGTL

SEQ ID NO: 6 - protein, 24 aa, Artificial Sequence
  synthetic sequence
LRELDLAQEKTGWFALGSRHCGRT

SEQ ID NO: 7 - protein, 23 aa, Artificial Sequence
  synthetic sequence
YKKAGSGIPLRMNSLFRKRNKGK

SEQ ID NO: 8 - protein, 23 aa, Artificial Sequence
  synthetic sequence
VFSDLKNESGQEDKKRLLLGLDR

SEQ ID NO: 9 - protein, 22 aa, Artificial Sequence
  synthetic sequence
VFSDLKNESGQEDKKRLLLGLD

SEQ ID NO: 10 - protein, 23 aa, Artificial Sequence
  synthetic sequence
DGEDEGYFQELLGSVNSLLKELR

SEQ ID NO: 11 - protein, 22 aa, Artificial Sequence
  synthetic sequence
DGEDEGYFQELLGSVNSLLKEL

SEQ ID NO: 12 - protein, 23 aa, Artificial Sequence
  synthetic sequence
LEKEKLQARQQQLLKKIEHARTC

SEQ ID NO: 13 - protein, 20 aa, Artificial Sequence
  synthetic sequence
LEKEKLQARQQQLLKKIEHA

SEQ ID NO: 14 - protein, 23 aa, Artificial Sequence
  synthetic sequence
GPGPNGQRFCINSAALKFKPRKH

SEQ ID NO: 15 - protein, 23 aa, Artificial Sequence
  synthetic sequence
LRNAQMKNAIRKLCSRKAISSVK

SEQ ID NO: 16 - protein, 23 aa, Artificial Sequence
  synthetic sequence
LRNAQMKNAIRKLCSRKAISSDK

SEQ ID NO: 17 - protein, 23 aa, Artificial Sequence
  synthetic sequence
RRSCNDCQQMIVKLGGGTAIAAS

SEQ ID NO: 18 - protein, 23 aa, Artificial Sequence
  synthetic sequence
RRSCNDCQQMIVKLGGGTAIADS

SEQ ID NO: 19 - protein, 23 aa, Artificial Sequence
  synthetic sequence
QKSRKVDSKSLFHYRQYSAGKAA

SEQ ID NO: 20 - protein, 23 aa, Artificial Sequence
  synthetic sequence
QKSRKVDSKSLFHYRQYSAGKDD

SEQ ID NO: 21 - protein, 28 aa, Artificial Sequence
  synthetic sequence
QKSRKVDSKSLFHYRQYSAGKAAKASTN

SEQ ID NO: 22 - protein, 23 aa, Artificial Sequence
  synthetic sequence
REIQGIFFKEDSHKESNDCSCGG

SEQ ID NO: 23 - protein, 23 aa, Artificial Sequence
  synthetic sequence
SLTDVALEHHEECDCVCRGSTGG

SEQ ID NO: 24 - protein, 23 aa, Artificial Sequence
  synthetic sequence
RRLDGKEEDEKMSRASDRFRGLR

SEQ ID NO: 25 - protein, 19 aa, Artificial Sequence
  synthetic sequence
TRGVEEVAEGVVLLRRRGN

SEQ ID NO: 26 - protein, 10 aa, Artificial Sequence
  synthetic sequence
GIPLRNLGIR

SEQ ID NO: 27 - protein, 15 aa, Artificial Sequence
  synthetic sequence
GIPLRQRKLQRTSRG

SEQ ID NO: 28 - protein, 15 aa, Artificial Sequence
  synthetic sequence
GIPLRQRKLQRTSRA

SEQ ID NO: 29 - protein, 17 aa, Artificial Sequence
  synthetic sequence
GIPLRPHKRLLKGSQYG

SEQ ID NO: 30 - protein, 18 aa, Artificial Sequence
  synthetic sequence
TRGVEEVAEGVVLLRRRG

SEQ ID NO: 31 - protein, 6 aa, Artificial Sequence
  synthetic sequence
TRRRGN

SEQ ID NO: 32 - protein, 4 aa, Artificial Sequence
  synthetic sequence
RRRG

SEQ ID NO: 33 - protein, 115 aa, Artificial Sequence
  synthetic sequence
QIFVKTLTGKTITLEVESSDTIDNVKSKIQDKEGIPPDQQRLIFAGKQLEDGRTLSDYNIQKESTLHLVLRLRGGRHGSGAWLLPVSLVRRRTTLAPNTQTASPRALADSLMQRS

SEQ ID NO: 34 - protein, 21 aa, Artificial Sequence
  synthetic sequence
DDLLLPQDVEEFFEGPSEALR

SEQ ID NO: 35 - protein, 7 aa, Artificial Sequence
  synthetic sequence
ALAPYIP

SEQ ID NO: 36 - protein, 14 aa, Artificial Sequence
  synthetic sequence
KFERQKILDQRFFE

SEQ ID NO: 37 - protein, 21 aa, Artificial Sequence
  synthetic sequence
PLSSSVPSQKTYQGSYGFRLG

SEQ ID NO: 38 - protein, 4 aa, Artificial Sequence
  synthetic sequence
GSYG

SEQ ID NO: 39 - protein, 10 aa, Artificial Sequence
  synthetic sequence
DVQKADVSST

SEQ ID NO: 40 - protein, 8 aa, Artificial Sequence
  synthetic sequence
SPDSSTSP

SEQ ID NO: 41 - protein, 4 aa, Artificial Sequence
  synthetic sequence
RRRG

SEQ ID NO: 42 - protein, 5 aa, Artificial Sequence
  synthetic sequence
DINNN

(19) United States
(12) Patent Application Publication
(10) Pub. No.: US 2024/0132560 A1
(43) Pub. Date: Apr. 25, 2024

(54) CONDITIONAL DEGRADATION OF PROTEINS THAT ARE LOCALIZED AT THE PLASMA MEMBRANE

(71) Applicant: The Regents of the University of California, Oakland, CA (US)

(72) Inventors: Andrew H. NG, San Francisco, CA (US); Matthew KIM, San Francisco, CA (US); Hana EL-SAMAD, San Francisco, CA (US)

(21) Appl. No.: 18/278,377

(22) PCT Filed: Mar. 13, 2022
(86) PCT No.: PCT/US2022/020213

§ 371 Date: Aug. 21, 2023

Related U.S. Application Data
(60) Provisional application No. 63/161,307, filed on Mar. 15, 2021.

(51) Int. Cl. C07K 14/47; C07K 14/705

(52) U.S. Cl. CPC C07K 14/4703; C07K 14/705; C07K 2317/622; C07K 2319/03; C07K 2319/95

(57) ABSTRACT

Provided herein is a fusion protein comprising: (a) an extracellular domain comprising a first binding moiety that is capable of specifically binding to a first cell surface marker: (b) a transmembrane domain: and (c) an intracellular domain comprising: i. a first dimerization domain that specifically binds to a corresponding target dimerization domain in a target protein: and ii. a degradation domain, wherein the degradation domain is a degron or E3 ligase-recruiting domain. Protein circuits. cells and methods that make use of the fusion protein are also provided.

### CROSS-REFERENCING

This application claims the benefit of U.S. provisional application Ser. No. 63/161,307, filed on Mar. 15, 2021, which application is incorporated by reference herein for all purposes.

### GOVERNMENT RIGHTS

This invention was made with government support under grant no. HR0011-16-2-0045 awarded by Defense Advanced Research Projects Agency. The government has certain rights in the invention.

### BACKGROUND

Although some cell therapies have demonstrated remarkable therapeutic responses and benefits for patients with some diseases, development of effective cell-based therapies for other diseases remains a challenge, in large part due to the difficulty in delivering therapeutics only to a specific tissue. For example, some treatments that kill cells expressing a marker for cancer may be detrimental to other, normal, cells that normally express that marker. As such, administrating a therapy that targets diseased cells in one tissue can often cause side-effects in another. Because of this, the clinical use of several therapies that have enormous potential has been typically limited by off-site effects, rather than the on-site effects. This problem is exacerbated in immune cell-based therapies because many of those therapies are extremely potent.

In order to avoid off-site effects, it would be desirable to “turn off” therapeutic cells in a particular in a tissue. This disclosure addresses this issue and others.

### SUMMARY

This disclosure provides a way to degrade transmembrane receptor proteins, e.g., a chimeric antigen receptor (CAR), in an antigen-dependent manner. The method makes use of a fusion protein that contains an extracellular protein binder (such as a scFv) linked to a transmembrane domain and a degradation domain and a dimerization domain, which in some cases, may have a low affinity for its target. The combined protein degradation domain and dimerization domain may be referred to as a “synthetic targeter of ubiquitination and degradation”, or “STUD” for short in this disclosure. In some cases, the STUD's affinity for its target is lowered such that it will interact only very weakly with its target. In some cases, the target protein may be a CAR and, in these embodiments, the presence of two proteins in the surface of another cell—a protein to which the fusion protein binds and a protein to which the CAR binds—drives colocalization of the two membrane proteins, and drives interaction of the STUD with the CAR via increased local concentration of the two proteins, overcoming the weak affinity binder. This system enables NOT logic gating in cell therapies. Recognition of an off-target (healthy) antigen via the coSTUD can drive degradation of a CAR and thus prevent off-tumor toxicity of cell therapies.

This system, which may be referred to as the “coSTUDs” system can implement NOT logic in cell therapies, enabling targeting of antigen A (disease antigen) and NOT antigen B (healthy antigen). coSTUDs could be used to regulate conditional regulation of any membrane protein, synthetic or endogenous, depending on the targeting domain used on the STUD. This means that coSTUDs could be used to regulate the activity of CARS, SynNotch, or even endogenous signaling receptors such as TCRs or PD-1.

In some embodiments, the fusion protein comprises: (a) an extracellular domain comprising a first binding moiety that is capable of specifically binding to a first cell surface marker; (b) a transmembrane domain; and (c) an intracellular domain comprising: i. a first dimerization domain that specifically binds to a corresponding target dimerization domain in a target protein; and ii. a degradation domain, wherein the degradation domain is a degron or E3 ligase-recruiting domain. Protein circuits, cells and methods that make use of the fusion protein are also provided.

In some embodiments, the fusion protein may be employed as part of a protein circuit to conditionally degrade a target protein that, in many embodiments, may be localized to the plasma membrane. In these embodiments, the method may comprise introducing a first cell to a second cell, e.g., by mixing cells in vitro or by administering the first cell into a subject. In this method: (a) the first cell comprises i. the fusion protein as well as ii. a target protein, wherein the target protein comprises: an extracellular binding domain comprising a second binding region that is capable of specifically binding to a second cell surface marker, a transmembrane domain and an intracellular domain that comprises a target dimerization domain to which the first dimerization domain of the fusion protein binds with a low affinity, and (b) the second cell comprises, the first and second cell surface markers. In this method, binding of the fusion protein and the target protein to molecules on the same cell brings the fusion protein and the target protein in closer proximity, which increases the rate of binding between the proteins (via the dimerization domains), which, in turn, increases the rate of degradation of the target protein.

In one hypothetical example, the therapeutic cell could be an immune cell (e.g., a T cell), the fusion protein could recognize a liver antigen and the target protein could be a chimeric antigen receptor (CAR) that recognizes a breast cancer antigen. In this example, the immune cells would become activated only when they are in contact with cells that express the breast cancer antigen but not the liver antigen. If the breast cancer antigen and liver antigen are expressed on the same cell, then, in theory, the fusion protein and the CAR should dimerize with one another at a higher rate which, in turn, will cause the CAR to be degraded.

Cell therapies developed using coSTUDs may have more potent NOT (inhibitory) activity compared to current approaches such as inhibitory CARs (iCARs), SUPRA CAR, and coLOCKR. These other technologies have previously been shown to provide only moderate reduction of CAR T cell activity. For example, iCARs activate negative signaling through ITIMs from PD-1 and other checkpoint inhibitors to reduce T cell activation. However, CAR signaling can be too powerful and can easily overcome these inhibitory signals. Strategies such as SUPRA CAR and coLOCKR rely on protein competition to prevent protein binding and activation of CARs. However, because it can be difficult to tune the affinities in these systems, full repression of protein binding while maintaining activation is challenging. Finally, SynNotch NOT gate strategies that activate cell apoptotic markers such as tBID can be powerful but also irreversibly kill the therapeutic cell itself. The current system has the potential to improve on all of these technologies because it prevents signal transduction by protein degradation and it does not kill the cell when it is activated.

These and other advantages may become apparent in view of the following discussion.

### BRIEF DESCRIPTION OF THE FIGURES

The skilled artisan will understand that the drawings, described below, are for illustration purposes only. The drawings are not intended to limit the scope of the present teachings in any way.

FIG. 1 schematically illustrates an example of the present fusion protein.

FIGS. 2A-2C schematically illustrates the principle upon which some embodiments of the present method work. FIG. 2A is an illustration showing that truncated SynZIPs have lower affinity interactions then full length SynZIPs. FIG. 2B is an illustration showing that a pair of fusion proteins are more likely to dimerize if they are brought into proximity. As illustrated in FIG. 2C, the proximity-dependent dimerization system of FIG. 2B can be used to conditionally degrade a signaling protein at the plasma membrane (e.g., a chimeric antigen receptor).

FIG. 3: Lysine to arginine substitution significantly improves STUD activity. Either a GFP nanobody (vhhGFP4) or SynZIP (SZ18) were used to target a GFP (or in the case of the SZ18 STUD, GFP-SZ17. SZ17 and SZ18 form a cognate pair). GFP % Degradation was measured compared to GFP fluorescence in the absence of the STUD.

FIG. 4: MG132 proteasome inhibitor confirms that the effect of STUD is mediated by the proteasome. Primary human CD4+T cells expressing different variants of the GFP nanobody STUD were few 5 uM MG132 and fluorescence was measured at 1 and 3 hours post induction. The mutant nanobody was the only experimental group that exhibited an increase in fluorescence over time, suggesting the effect of the STUD is mediated by protein degradation through the proteasome.

FIG. 5: Optimizing STUD activity via linker modification in Jurkat cells. A variety of flexible (GS) and rigid linkers were tested between the SynZIP targeting domain and degron on the STUD. Flexible linkers generally outperformed rigid linkers, and in particular the 5xGS linker produced the greatest degradation.

FIG. 6: Design of a circuit to test STUD induced degradation of a synthetic transcription factor. VPR-NS3-ZF3 drives activation of the pZF3(8x)_ybTATA promoter in response to induction with GRZ. Three different circuit configurations were explored. This circuit provides feedback, where STUD is driven off the pZF3 promoter, GFP alone, where no STUD is expressed, and Constitutive STUD, where the STUD is expressed off the pPGK promoter

FIG. 7: ZF3 circuit dose responses demonstrate the functionality of the soluble STUD to degrade a transcription factor. The circuits shown in FIG. 3 were transduced into Jurkat cells and induced with a range of GRZ concentrations to activate the TF. GFP fluorescence was measured 72 hours later.

FIG. 8: Testing the ability of soluble STUDs to target a CAR-SZ17 fusion for degradation in Jurkat cells. Four different linker lengths between the SynZIP18 on the STUD and degron were tested. A control where the degron was directly fused to the CAR generated the most degradation.

FIG. 9: Design of membrane targeting STUD. DAP10 extracellular domain (ECD) contains a signal sequence that traffics the protein in the membrane. The CD8 transmembrane domain (TMD) embeds in membrane and is linked to the soluble STUD via a linker.

FIG. 10: Degradation of CAR in primary human CD4+ T cells. Rigid15 linker between CD8 TMD and soluble STUD mediated the greatest amount of CAR degradation as measured by staining for the myc-tag present on the CAR and flow cytometry.

FIG. 11: Degradation of SynNotch in primary human CD4+ T cells. Rigid15 linker between CD8 TMD and soluble STUD mediated the greatest amount of SynNotch degradation as measured by staining for the myc-tag present on the SynNotch and flow cytometry.

### DEFINITIONS

Unless otherwise defined, all technical and scientific terms used herein have the same meaning as commonly understood by one of ordinary skill in the art to which this invention belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

“Operably linked” refers to a juxtaposition wherein the components so described are in a relationship permitting them to function in their intended manner. For instance, a promoter is operably linked to a coding sequence if the promoter affects its transcription or expression.

A “vector” or “expression vector” is a replicon, such as plasmid, phage, virus, or cosmid, to which another DNA segment, i.e. an “insert”, may be attached so as to bring about the replication of the attached segment in a cell.

“Heterologous,” as used herein, means a nucleotide or polypeptide sequence that is not found in the native (e.g., naturally-occurring) nucleic acid or protein, respectively.

The terms “antibodies” and “immunoglobulin” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. The antibodies can also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies.

As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (VHH) derived from naturally occurring heavy chain antibody and is known to the person skilled in the art. They are derived from heavy chain only antibodies, seen in camelids (Hamers-Casterman et al., 1993; Desmyter et al., 1996). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a VHH antibody.

“Antibody fragments” comprise a portion of an intact antibody, for example, the antigen binding or variable region of the intact antibody. Examples of antibody fragments include Fab, Fab′, F(ab′)2, and Fv fragments; diabodies; linear antibodies (Zapata et al., Protein Eng. 8(10): 1057-1062 (1995)); domain antibodies (dAb; Holt et al. (2003) Trends Biotechnol. 21: 484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)2 fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CHI domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the VH and VL domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

The term “binding” refers to a direct association between two molecules, due to, for example, covalent, electrostatic, hydrophobic, and ionic and/or hydrogen-bond interactions, including interactions such as salt bridges and water bridges. In some cases, the first member of a specific binding pair present in the extracellular domain of a chimeric Notch receptor polypeptide of the present disclosure binds specifically to a second member of the specific binding pair.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

The terms “chimeric antigen receptor” and “CAR”, used interchangeably herein, refer to artificial multi-module molecules capable of triggering or inhibiting the activation of an immune cell which generally but not exclusively comprise an extracellular domain (e.g., a ligand/antigen binding domain), a transmembrane domain and one or more intracellular signaling domains. The term CAR is not limited specifically to CAR molecules but also includes CAR variants. CAR variants include split CARs wherein the extracellular portion (e.g., the ligand binding portion) and the intracellular portion (e.g., the intracellular signaling portion) of a CAR are present on two separate molecules. CAR variants also include ON-switch CARs which are conditionally activatable CARs, e.g., comprising a split CAR wherein conditional hetero-dimerization of the two portions of the split CAR is pharmacologically controlled. CAR variants also include bispecific CARs, which include a secondary CAR binding domain that can either amplify or inhibit the activity of a primary CAR. CAR variants also include inhibitory chimeric antigen receptors (iCARs) which may, e.g., be used as a component of a bispecific CAR system, where binding of a secondary CAR binding domain results in inhibition of primary CAR activation. CAR molecules and derivatives thereof (i.e., CAR variants) are described, e.g., in PCT Application No. US2014/016527; Fedorov et al. Sci Transl Med (2013); 5(215): 215ra172; Glienke et al. Front Pharmacol (2015) 6: 21; Kakarla & Gottschalk 52 Cancer J (2014) 20(2): 151-5; Riddell et al. Cancer J (2014) 20(2): 141-4; Pegram et al. Cancer J (2014) 20(2): 127-33; Cheadle et al. Immunol Rev (2014) 257(1): 91-106; Barrett et al. Annu Rev Med (2014) 65: 333-47; Sadelain et al. Cancer Discov (2013) 3(4): 388-98; Cartellieri et al., J Biomed Biotechnol (2010) 956304; the disclosures of which are incorporated herein by reference in their entirety.

As used herein, the terms “treatment,” “treating,” “treat” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which can be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), lagomorphs, etc. In some cases, the individual is a human. In some cases, the individual is a non-human primate. In some cases, the individual is a rodent, e.g., a rat or a mouse. In some cases, the individual is a lagomorph, e.g., a rabbit.

The term “marker” refers to a protein that is on the surface of another cell. A marker may be a cell surface receptor, an epitope in a cell surface protein or a cell-surface ligand.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

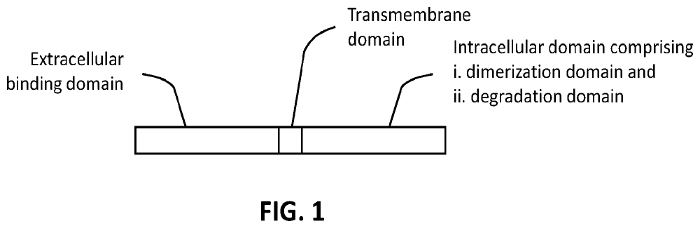

As noted above, this disclosure provides a fusion protein comprising (a) an extracellular domain comprising a first binding moiety (e.g., a scFv, a nanobody or a ligand for a cell-surface receptor) that is capable of specifically binding to a cell surface marker, (b) a transmembrane domain; and (c) an intracellular domain comprising: i. a first dimerization domain that specifically binds to a corresponding target dimerization domain in a target protein; and ii. a degradation domain, wherein the degradation domain is a degron or E3 ligase-recruiting domain. This fusion protein is schematically illustrated in FIG. 1.

Depending on how the fusion protein is designed, the extracellular domain may be N-terminal N-terminal or C-terminal to the intracellular domain, and, likewise, the dimerization domain may be N-terminal N-terminal or C-terminal to the degradation domain. In any embodiment, the fusion protein may optionally contain a linker any of the component parts, e.g., the dimerization domain and the degradation domain. In a cell, binding of the fusion protein to a target protein via the dimerization domain induces degradation of the target protein. Degradation may be ubiquitination-mediated or not ubiquitination-mediated, depending on which degradation domain is used. The various components parts of the fusion protein are described below.

### First Binding Moieties

The extracellular domain of the fusion protein comprises a first binding moiety that specifically binds to a cell surface marker on a cell. In some embodiments, the first binding moiety could be a scFv, a nanobody (i.e., a single chain antibody derived from a camel or shark antibody variable domain), or a ligand for a cell-surface receptor, although other types of binding moieties can be used in certain circumstances. For example, a cAb VHH (camelid antibody variable domain), IgNAR VH (shark antibody variable domain) and/or sdAb VH (single domain antibody variable domain) and “camelized” antibody variable domains (including humanized versions of the same) could be employed. First binding moieties include, for example, antibody binding domains that bind to cell surface antigens, ligands that bind to cell surface receptors and receptors that bind to ligands. Other types of binding domains could be used in certain cases.

As will be apparent from the description that follows below, the cell surface marker to which the first binding moiety binds may vary depending on the cell surface marker to which the first binding moiety binds and how the fusion protein is going to be used.

### Transmembrane Domains

The fusion protein has a transmembrane domain. Suitable transmembrane domains include those of CD8, CD4, CD3 zeta, CD28, CD134, CD7, although there are thousands of others that one could use. As would be apparent, the nucleic acid encoding such a fusion protein may additionally comprise a signal peptide.

### Linkers

In some embodiments, the fusion protein may further comprise a linker, between any two component parts of the fusion protein. A peptide linker can vary in length of from about 3 amino acids (aa) or less to about 200 aa or more, including but not limited to e.g., from 3 aa to 10 aa, from 5 aa to 15 aa, from 10 aa to 25 aa, from 25 aa to 50 aa, from 50 aa to 75 aa, from 75 aa to 100 aa, from 100 aa to 125 aa, from 125 aa to 150 aa, from 150 aa to 175 aa, or from 175 aa to 200 aa. A peptide linker can have a length of from 3 aa to 30 aa, e.g., 3, 4, 5, 6, 7, 8, 9, 10, 11, 12, 13, 14, 15, 16, 17, 18, 19, 20, 21, 22, 23, 24, 25, 26, 27, 28, 29, or 30 aa. A peptide linker can have a length of from 5 aa to 50 aa, e.g., from 5 aa to 40 aa, from 5 aa to 35 aa, from 5 aa to 30 aa, from 5 aa to 25 aa, from 5 aa to 20 aa, from 5 aa to 15 aa or from 5 aa to 10 aa.

Suitable linkers can be readily selected and can be of any of a number of suitable lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and can be 1, 2, 3, 4, 5, 6, or 7 amino acids.

Exemplary linkers include glycine polymers (G)n, glycine-serine polymers, glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)).

### Dimerization Domains

The dimerization domain of the fusion protein (the “first” dimerization domain) may be any engineered domain that orthogonally binds to another dimerization domain (the “target” dimerization domain) in a target protein (i.e., without binding to other proteins). Pairs of dimerization domains include, but are not limited to, the helix-turn-helix-based designed heterodimers (DHDs) described in Chen et al (Nature. 2019 565: 106-111), the heterospecific synthetic coiled-coil synthetic leucine zippers (synZIPs) described in, e.g., Thompson et al. (ACS Synth. Biol. 2012 1: 118-29), Reinke et al. (JACS 2010 132:6025-31) and (Cho et al Cell 2018 173: 1426-1438), miniproteins (Nature 2017 550: 74-79), intrabodies (Chen et al Human Gene Therapy 1994 5: 595-601). scFvs, nanobodies, Fabs, DARPins and monobodies could also be used, among many others. For example, if synZIPs are used, one dimerization domain may be BZip (RR) and the other one may be AZip (EE). SYNZIP 1 to SYNZIP 48, and BATF, FOS, ATF4, ATF3, BACHI, JUND, NFE2L3, and HEPTAD may be used in some cases.

In some embodiments, the first dimerization domain binds to the target dimerization with a low affinity. In these embodiments, the first dimerization domain may bind to the target dimerization with a Kd of greater than 10 nM (e.g., a K the range of 50 nM to 1000 nM, or at least 100 nM to 500 nM), as measured by the method of Thompson (ACS Synth. Biol. 2012 1: 118-29).

Dimerization domains that bind to one another with a low affinity can be engineered from high affinity interactions relatively straightforwardly. For example, domains that interact with one another with a high affinity may be modified to decrease the affinity of the interaction. Such methods include, but are not limited to e.g., random (untargeted) and targeted (directed) mutagenesis, alanine scanning, and screening (e.g., phage display, etc.) methods. For example, synZIPs are around 30 amino acids in length and are composed of eight a-helical turns, with 5 leucines that spaced every 7 aa. To decrease the affinity of synZIP, one could remove 7 residues at a time from the ends to remove a single heptad repeat at a time. As such, if a low affinity synZIP is used one of the synthetic leucine zipper domains may have up to seven a-helical turns (e.g., 5, 6, or 7 turns), not the full complement of eight a-helical turns.

In some instances, the affinity of a dimerization pair may be assessed, estimated, and/or quantitated in various ways. For example, in some instances, affinity may be assessed, estimated, and/or quantitated by a biochemical or biophysical method. Useful methods for assessing, estimating, and/or determining absolute and/or relative and/or estimated affinities may include but are not limited to e.g., affinity electrophoresis, bimolecular fluorescence complementation (BiFC), bio-layer interferometry, co-immunoprecipitation, dual polarization interferometry (DPI), dynamic light scattering (DLS), flow-induced dispersion analysis (FIDA), fluorescence correlation spectroscopy, fluorescence polarization/anisotropy, fluorescence resonance energy transfer (FRET), isothermal titration calorimetry (ITC), microscale thermophoresis (MST), phage display, proximity ligation assay (PLA), quantitative immunoprecipitation combined with knock-down (QUICK), rotating cell-based ligand binding assay, static light scattering (SLS), single colour reflectometry (SCORE), surface plasmon resonance (SPR), tandem affinity purification (TAP), and the like.

### Degrons

Degrons are relatively short (typically under 100 amino acids) sequences that, when they are present in a protein, target that protein for degradation. Degrons include ubiquitin-dependent degrons and ubiquitin-independent degrons. Examples of degrons include ubiquitin (which is approximately 76 amino acids in length), PEST sequences (which are approximately 10 to 60 amino acids in length and are rich in P (proline), E (glutamate), S (serine), and T (threonine)), N-degrons (which are short N-terminal sequences), C degrons (which are short N-terminal sequences), unstructured initiation sites and short sequences rich in acceptor lysines. Degrons are diverse in sequence and have been extensively reviewed (see, e.g., Varshavsky, Proc. Natl. Acad. Sci. 2019 116: 358-366; Varshavsky, Protein Sci. 2011 20: 1298-1345; Natsume et al., Annu Rev. Genet 2017 51: 83-102; Rechsteiner et al., Trends Biochem Sci. 1996 21: 267-271; Herbst et al., Oncogene 2004 23: 3863-3871;

Prakash, Nat. Struct. Mol. Biol. 2004 11: 830-837; Guharoy et al., Nat. Commun. 2016 7: 10239 and Chassin et al. Nature Comm. 2019 10).

The “Bonger”-type degron (which has the sequence RRRG the C terminus or one amino acid away from the C terminus) may be used in any fusion protein, although there are many alternatives that could be used instead.

Examples of C-degrons suitable for use in a fusion protein are listed below (see Koren et al., Cell 2018 173: 1622-1635):

| | | | SEQ |
|---|---|---|---|
| Name | Sequence | Motif | ID NO: |
| fRA68_EMID1 | RGKRGGHATNYRIVAPRSRDERG* | RG* | 1 |
| fRA69_CHGA | ESLSAIEAELEKVAHQLQALRRG* | RG* | 2 |
| fRA70_MAGEA3 | KISGGPHISYPPLHEWVLREGEE* | EE* | 3 |
| fRA71_MAGEA3EEtoAA | KISGGPHISYPPLHEWVLREGAA* | EE* to | 4 |
| | | Ax/A* | |
| fRA72_PIK3C2B | LRELDLAQEKTGWFALGSRSHGTL* | RxxGxx* | 5 |
| fRA73_PXN | LRELDLAQEKTGWFALGSRHCGRT* | RxxGxx* | 6 |
| fRA74_Peptide35 | YKKAGSGIPLRMNSLFRKRNKGK* | RxxGxx* | 7 |
| fRA75_CDK5R1 | VFSDLKNESGQEDKKRLLLGLDR* | R* motif, | 8 |
| fRA76_CDK5R1trunc | VFSDLKNESGQEDKKRLLLGLD* | R | 9 |
| | | truncated, | |
| fRA77_SIL1 | DGEDEGYFQELLGSVNSLLKELR* | R* | 10 |
| fRA78_SIL1trunC | DGEDEGYFQELLGSVNSLLKEL* | R | 11 |
| | | truncated, | |
| fRA79_N-Myc | LEKEKLQARQQQLLKKIEHARTC* | Rxx* | 12 |
| fRA80_N-Myctrunc | LEKEKLQARQQQLLKKIEHA* | Rxx* to | 13 |
| | | A*, | |
| fRA81_MSRB2 | GPGPNGQRFCINSAALKFKPRKH* | Rxx* | 14 |
| fRA82_OR4C13 | LRNAQMKNAIRKLCSRKAISSVK* | Vx* motif | 15 |
| fRA83_OR4C13Dmut | LRNAQMKNAIRKLCSRKAISSDK* | Vx to Dx | 16 |
| fRA84_SREBF2 | RRSCNDCQQMIVKLGGGTAIAAS* | Ax* | 17 |
| fRA85_SREBF2-Dmut | RRSCNDCQQMIVKLGGGTAIADS* | Ax | 18 |
| fRA86_CPS1 | QKSRKVDSKSLFHYRQYSAGKAA* | AA* | 19 |
| fRA87_CPS1DDmut | QKSRKVDSKSLFHYRQYSAGKDD* | AA to DD, | 20 |
| fRA88_CPS1CText | QKSRKVDSKSLFHYRQYSAGKAAKASTN* | AA Ct | 21 |
| fRA89_EPHB2 | REIQGIFFKEDSHKESNDCSCGG* | GG | 22 |
| fRA90_PDGFC | SLTDVALEHHEECDCVCRGSTGG* | GG | 23 |
| fRA91_ASCC3 | RRLDGKEEDEKMSRASDRFRGLR* | RG/R* | 24 |
| | | dual | |
| RA2102_degBon1 | TRGVEEVAEGVVLLRRRGN* | Rxx*/RxxG | 25 |
| RA2106_Clone1 | (GIPLR) NLGIR* | RG/R* | 26 |
| | | dual | |
| RA2107_Clone6 | (GIPLR) QRKLQRTSRG* | RG* | 27 |
| RA2108_Clone6GtoA | (GIPLR) QRKLQRTSRA* | RG* to A*, | 28 |
| RA2109_Clone8 | (GIPLR) PHKRLLKGSQYG* | RG*-like | 29 |

Further examples of C-degrons suitable for use in a fusion protein are listed below (see Bonger, Nat Chem Biol 7, 531-537):

| RA2103_degBon2 | TRGVEEVAEG | dual motif | SEQ ID NO: |
|---|---|---|---|
| | VVLLRRRG* | (RG*) | 30 |
| RA2104_degBon3 | TRRRGN* | stronger | SEQ ID NO: |
| | | variant | 31 |
| RA2105_degBon4 | RRRG* | strongest | SEQ ID NO: |
| | | variant | 32 |

One example of an N-degron suitable for use in a fusion protein is listed below (see Bachmair et al. Cell 1989 56. 1019-1032). This sequence is a fusion of Ubiquitin and N terminus of B-gal.

| | | |
|---|---|---|
| Ubi-R | QIFVKTLTGKTITLEVESSDTIDNVKSKIQDKEGIP | SEQ ID |
| | PDQQRLIFAGKQLEDGRTLSDYNIQKESTLHLVLRL | NO: 33 |
| | RGGRHGSGAWLLPVSLVRRRTTLAPNTQTASPRALA | |
| | DSLMQRS | |

One example of a PEST sequence suitable for use in a fusion protein is listed below (see Rogers et a; Science 1986 234: 364-8). There are many examples of PEST sequences.

| | | | SEQ |
|---|---|---|---|
| Name | Sequence | Origin | ID NO |
| p53 PEST | DDLLLPQDVEEFFEGPSEALR | p53 | 34 |

Further examples of degrons that could be employed are shown below. These sequences are disclosed in Hon et al. (Nature 2002 417: 975-8), Fan et al. (Nat. Neurosci. 2014 17: 471-480), Gu et al. (Molecular and Cellular Biology 2000 20: 1243-1253), Melvin et al. (Analyst 2016 141:570-8) and Zhang et al. (Developmental Cell 2019 48: 329-344).

| | | | | SEQ |
|---|---|---|---|---|
| | | | E3 | ID |
| Name | Sequence | Origin | Ligase | NO: |
| VHLdeg | ALAPYIP | HIF-1a | VHL | 35 |
| CMAdeg | KFERQKILDQ | RNaseA- | Lysosome | 36 |
| | RFFE | hsc70- | | |
| | | hemoglobin | | |
| MDM2deg | PLSSSVPSQK | p54(92-112) | MDM2 | 37 |
| | TYQGSYGFRL | | | |
| | G | | | |
| MDM2(short) | GSYG | p54(92-112) | MDM2 | 38 |
| deg | | | | |
| SPOP(2)deg | DVQKADVSST | SRC3 | SPOP | 39 |
| SPOP(3)deg | SPDSSTSP | Nanog | SPOP | 40 |
| BONGERdeg | RRRG | Synthetic | Unknown | 41 |
| iNOSdeg | DINNN | iNOS | Unknown | 42 |

In this fusion protein, the degron works in trans, meaning that the target protein that is degraded is a different protein, i.e., the protein that the fusion protein (which contains the degron) binds to.

### E3 Ligase Recruiting Domains

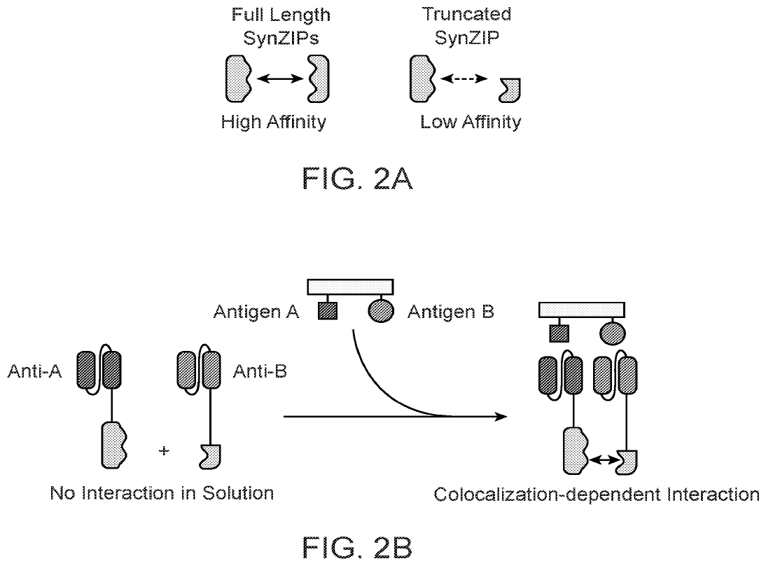

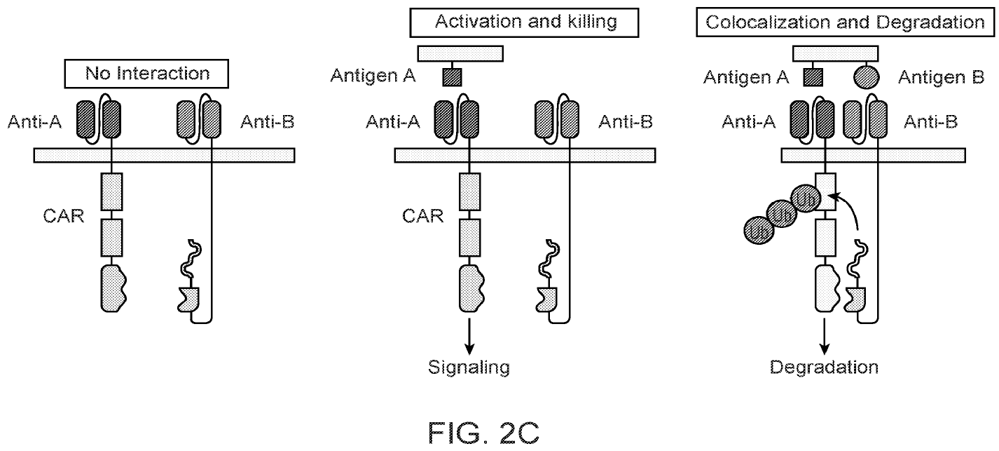

In the cell, the target-binding domain of the fusion protein binds to a target protein and recruits it into an E3-ligase complex, thereby causing the target to be ubiquitinated and degraded. In some embodiments, the E3 ligase recruiting domain of the fusion protein may interact with an E3 ligase directly or indirectly. In these embodiments, the E3 ligase is endogenous to the cell. FIG. 2 illustrates some of the current models of how substrates are recruited for degradation. As shown in panels A, B, D, E and F, many complexes contain an adapter protein (e.g., Skp1, Elongin B/C or DDB1) that links the E3 ligase (a cullin) to a protein that binds to the substrate. The protein that binds to the substrate is referred to as a “receptor” (and may be an F-box protein, VHL-box protein, DCAF, SOCS, for example). In one model (c), the receptor binds directly to the E3 ligase. The degradation domain of a fusion protein can contain any of the interaction domains shown in FIG. 2 (e.g., the E3 ligase interaction domain of an adapter protein or receptor, or the adapter protein-interaction domain of a receptor). As would be apparent, if the fusion protein contains the E3 ligase interaction domain of an adapter protein or receptor, or the adapter protein-interaction domain of a receptor, then the fusion protein does not need to contain other parts of the protein. For example, if the target binding domain of the fusion protein is from an adapter protein, then the fusion protein does not need to contain the part of the adapter protein that binds to the receptor. In these embodiments, the fusion protein may contain the E3 ligase binding domain of an adapter protein but not the receptor binding domain of the adapter protein. Likewise, if the target binding domain of the fusion protein is from a receptor protein, then the fusion protein does not need to contain the part of the receptor protein that binds to the endogenous substrate. In these embodiments, the fusion protein may contain the adapter protein binding domain of a receptor but not the substrate binding domain of the receptor.

In some embodiments, the E3 ligase recruiting domain can directly interact with a Cullin protein. Examples of E3 ligase recruiting domains that directly interact with a Cullin protein may be found in E3 complex adapter proteins and in some substrate receptors (e.g., BTB). These complexes promote the transfer of ubiquitin from the E2 to the substrate, which targets the protein for degradation. Many complexes contain an adapter protein (e.g., SKP1 for CUL1 and CUL7, Elongin B/C for CUL2 and CUL5, BTB for CUL3 and DDB1 for CUL4A/b) as well as a receptor protein (F-box proteins for CUL1, VHL-box proteins for CUL2, DCAFs for CUL4A and 4B, SOCS for CUL5 and Fbx W8 for CUL7) and a RING protein (RB 1/2).

For example, an E3 ligase recruiting domain that directly interacts with an E3 ligase may have the Cullin binding region of an adapter protein, such as Skp1, ElonginB/C, or DDB 1 (as illustrated in FIG. 2). These Cullin binding regions have been studied in depth (see, e.g., Schulman Nature 2000 408: 381-386, Zheng et al. Nature 2002 416: 703 and Fischer Nature 2014 512: 49-53) and the sequence of these domains can be readily derived from these studies. For example, Skp1 and ElonginC have a conserved BTB/POZ domain that interacts with CUL1 and CUL2/5, respectively.

In another example, an E3 ligase recruiting domain that directly interacts with a Cullin protein may have a BTB domain. Examples of BTB domains can be found in substrate receptors that interact directly with CUL3. Examples of such substrate receptors that directly interact with CUL3 include SPOP and KLHL family (e.g., Keap1) members. These Cullin binding regions have been studied in depth (see, e.g., Stogios et al. Genome Biology 2005 6: R82, Zhuang et al. Molecular Cell 2009 36: 39-50 and Lee et al. Molecular Cell 2009 36: 131-140) and the sequence of these domains can be readily derived from these studies.

In other embodiments, the E3 ligase recruiting domain may indirectly interact with an E3 ligase protein. This interaction may be via an adapter protein. Examples of E3 ligase recruiting domains that indirectly interact with an E3 ligase may be found in some E3 substrate receptors (e.g., those receptors that interact with a Cullin via an adapter protein).

For example, an E3 ligase recruiting domain that indirectly interacts with an E3 ligase may have an F-box. Examples of F-box domains can be found in E3 substrate receptors that interact with Cullin-1 or Cullin-7 via Skp1. Canonical F-box proteins that bind Skp1 include FBWIA (beta-TRCP), Skp2, and Fbw7. The F box has been studied in depth (Su et al. Proc. Natl. Acad. Sci. 2003 100: 12729-12734; Schulman, Nature 2000 408: 381-386, Yumimoto Journal of Biological Chemistry 2-13 288: 28488-28502 and Skaar, Nature Reviews Molecular Cell Biology 2013 14: 369-381) and the sequence of this domain can be readily derived from these studies.

In another example, an E3 ligase recruiting domain may have a VHL- or SOCS-box. Examples of VHL- and SOCS-box domains can be found in E3 substrate receptors that interact Cullin-2 or Cullin-7 via Elongin B/C. Examples of F-box domains include members of suppressors of cytokine signaling (SOCS) family of proteins (e.g., Socs1, Socs3) as well as pVHL. The structure of these domains has been studied in depth (see. e.g., Liau et al. Nature Comm 2018 9: 1558, Stebbins et al. Science 1999 284: 455-461, Kamura, Genes & Development 2003 18: 3055-3065 and Linossi IUBMB Life 2012 64: 316-323) and the sequence of this domain can be readily derived from these studies.

In another example, an E3 ligase recruiting domain may have a WDXR motif. Examples of WDXR motifs can be found in E3 substrate receptors that interact with Cullin-4A or 4B, via DDB1. Examples of WDXR motifs include those of the DCAF family of proteins (e.g., DCAF1, DCAF9 and DDB2). DDB 1 interacts with CUL4 (similar to Skp1), and proteins such as DCAF1 provide the substrate recognition (similar to Skp2). DCAF1-type proteins use repeats of WD40 motifs, in which WDXR motifs are embedded, to bind to DDB1. The interactions between DDB11/WDXR proteins and E3 ligases have been studied in depth (see. e.g., Scrima et al. Cell 2008 135: 1213-1223, Yumimoto et al Journal of Biological Chemistry 2013 288: 28488-28502, Fischer et al Cell 2011 147: 1024-39, Fischer Nature 2014 512: 49-53, Schabla Journal of Molecular Cell Biology 2019 11: 725-735 and Jackson et al. Trends Biochem Sci. 2009 34: 562-570) and the sequence of this domain can be readily derived from these studies.

In alternative embodiments, the fusion protein could be a fusion between a target binding domain and an E3 ligase, such as one of the Cullins or E3 ubiquitin-protein ligase CHIP (see, e.g., Portnoff et al. J. Biol. Chem. 214 289: 7844 7855).

Finally, it may be possible to directly link the domain from Rbx1 that binds to E2 to a target binding domain. This fusion may still bind to the E3 ligase (as shown in FIG. 2) or it may bypass the E3 ligase if E2 can transfer ubiquitin onto substrates autonomously.

In any embodiment, the degradation domain, the target-binding domain and/or the linker may be selected or modified so that there are no lysines on the surface of the domain, thereby protecting the fusion protein from cis-ubiquitination and subsequent auto-degradation. In these embodiments, this domain may be designed by running a sequence through a structural prediction program, identifying lysines on the surface of a domain, and then changing the lysines to another residue (e.g., arginine, which is similar to lysine but not targeted by the ubiquitin ligase). In some embodiments, all of the lysines in one or more of the domains of the fusion protein may be modified to be arginines. In these embodiments, the fusion protein may be lysine free. In other embodiments, a subset of lysines (e.g., 1, 2, 3, 4, 5, 6 or 7 lysines) may be mutated to tune the balance of cis- versus trans-ubiquitination. These lysines may be identified based on their propensity for ubiquitination or surface accessibility. This strategy may be useful for tuning the activity of the protein degrader tool.

### Target Proteins

The target protein can be endogenous (i.e., native) to the cell or exogenous to the cell (i.e., expressed using recombinant means). In some embodiments, the target protein is a transmembrane protein. In these embodiments, the target protein may comprise: i. an extracellular binding domain comprising a second binding moiety that is capable of specifically binding to a second cell surface marker; ii. a transmembrane domain; and iii. an intracellular domain that comprises the target dimerization domain. Such target proteins may further comprises an effector region (e.g., a costimulatory domain) that is activated by binding of the extracellular binding domain to a target via the first binding region. Examples of such target proteins included chimeric antigen receptors (CARs), iCARs, synNotch receptors, chimeric costimulatory receptors (CCRs), chimeric T cell receptors, synthetic TCRs (e.g., the MART-1 TCR), switch receptors (i.e. receptors that comprise an endogenous extracellular binding domain of, e.g., an immune checkpoint inhibitor such as PD-1, and an intracellular costimulatory domain), GEMS (Scheller et al, Nat Chem Biol. 2018 14: 723-729) and MESAs (Daringer et al ACS Synth. Biol. 2014, 3, 12, 892-902). In other embodiments, the target protein may be associated with (i.e., bound to) the intracellular domain of a transmembrane protein such as PI3K, GRB2, TRAF1, ZAP70, TRAF6, IRAK1, IRAK4, MyD88, TIRAP, TRAM, TRIF, TBK1, a membrane-associated tyrosine kinase such as Lck, or any other component of the T cell signaling pathway such as LAT or Ras, etc.

In some embodiments, the target protein may be a therapeutic protein that, when expressed on the surface of an immune cell, activates the immune cell or inhibits activation of the immune cell when it binds to an antigen on the diseased cell. In these embodiments, the therapeutic protein may be a chimeric antigen receptor (CAR) or a T cell receptor (TCR). In these embodiments, the cell may be a T cell that expresses a CAR or TCR, where the CAR or TCR comprises an extracellular domain, a transmembrane region and an intracellular signaling domain; where the extracellular domain comprises a ligand or a receptor and the intracellular signaling domain comprises an ITAM domain, e.g., the signaling domain from the zeta chain of the human CD3 complex (CD3zeta), and, optionally, one or more costimulatory signaling domains, such as those from CD28, 4-1BB and OX-40. The extracellular domain contains a recognition element (e.g., an antibody or other target-binding scaffold) that enables the CAR to bind a target. In some cases, a CAR comprises the antigen binding domains of an antibody (e.g., an scFv) linked to T-cell signaling domains. In some cases, when expressed on the surface of a T cell, the CAR can direct T cell activity to those cells expressing a receptor or ligand for which this recognition element is specific. As an example, a CAR that contains an extracellular domain that contains a recognition element specific for a tumor antigen can direct T cell activity to tumor cells that bear the tumor antigen. The intracellular region enables the cell (e.g., a T cell) to receive costimulatory signals. The costimulatory signaling domains can be selected from CD28, 4-1BB, OX-40 or any combination of these. Exemplary CARs comprise a human CD4 transmembrane region, a human IgG4 Fc and a receptor or ligand that is tumor-specific, such as an IL13 or IL3 molecule. In these embodiments, activation of a CAR activates the immune cell.

Alternatively, the therapeutic protein may be an inhibitory immune cell receptor (iICR) such as an inhibitory chimeric antigen receptor (iCAR), wherein binding of the iICR to a marker on another cell inhibits activation of the immune cell on which the iICR is expressed. Such iICR proteins are described in e.g., WO2017087723, Fedorov et al. (Sci. Transl. Med. 2013 5: 215ra17) and other references cited above, which are incorporated by reference for that description and examples of the same. In some embodiments, an inhibitory immunoreceptor may comprise an intracellular immunoreceptor tyrosine-based inhibition motif (ITIM), an immunoreceptor tyrosine-based switch motif (ITSM), an Npx Y motif, or a YXXΦ motif. Exemplary intracellular domains for such molecules may be found in PD1, CTLA4, BTLA, CD160, KRLG-1, 2B4, Lag-3, Tim-3 and other immune checkpoints, for example. See, e.g., Odorizzi and Wherry (2012) J. Immunol. 188: 2957; and Baitsch et al. (2012) PLOSOne 7: e30852.

If the target protein is endogenous, then the dimerization domain of the fusion protein may contain a domain of a natural binding partner of the target protein, or another specific binding domain such as a nanobody or scFv.

If the target protein is exogenous, then in some cases the target protein can be engineered to contain a binding site for the dimerization domain of the fusion protein. In these embodiments, the target protein can be designed to contain an epitope tag (e.g., a hemagglutinin, FLAG, c-myc, ALFA, or V5 tag), and the like to which the dimerization domain binds. Alternatively, the target protein can be designed to contain a synthetic leucine zipper domain or any of the other domains described above, that heterodimerizes with a complementary synthetic leucine zipper domain in the fusion protein, as discussed above.

In some cases, binding of the fusion protein to the target protein may be conditional. In these embodiments, target binding domain of the fusion protein and the target protein may be engineered to only bind to one another in the presence of dimerization agent. Examples of pairs of protein domains that conditionally dimerize with one another include: FKBP and FKBP (which dimerize in the presence of rapamycin), FKBP and CnA (which dimerize in the presence of rapamycin), FKBP and cyclophilin (which dimerize in the presence of rapamycin), FKBP and FRG (which dimerize in the presence of rapamycin), GyrB and GyrB (which dimerize in the presence of coumermycin), DHFR and DHFR (which dimerize in the presence of methotrexate), DmrB and DmrB (which dimerize in the presence of AP20187), PYL and ABI (which dimerize in the presence of abscisic acid), Cry2 and CIB1 (which dimerize in the presence of blue light); GAI and GID1 (which dimerize in the presence of gibberellin) and a ligand-binding domain of a nuclear hormone receptor, and a co-regulator of the nuclear hormone receptor (which dimerize in the presence of a nuclear hormone, agonists thereof and antagonists thereof, e.g., tamoxifen). In embodiments in which rapamycin can serve a dimerizer, a rapamycin derivative or analog can also be used. In any embodiment, expression of the fusion protein may be inducible, tissue-specific, or constitutive. This may be done by operably linking the coding sequence for the fusion protein to an appropriate promoter.

### Cells

A therapeutic cell (e.g., a recombinant immune cell such as a CAR T, a Treg cell or stem cell) that expresses a fusion protein (i.e., contains an expression cassette comprising a promoter and, operably linked to the promoter, a coding sequence that encodes the fusion protein described above) is also provided. The therapeutic cell may be genetically modified to contain a nucleic acid comprising an expression cassette comprising a promoter and a coding sequence for the fusion protein as described above.

In some instances, a therapeutic cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or a progenitor thereof, obtained from an individual. As another example, the cell is a cytotoxic cell, or a progenitor thereof, obtained from an individual. As another example, the cell is a stem cell or progenitor cell obtained from an individual.

In some cases, the cell is an immune cell, e.g., a T cell, a B cell, a macrophage, a dendritic cell, a natural killer cell, a monocyte, etc. In some cases, the cell is a T cell. In some cases, the cell is a cytotoxic T cell (e.g., a CD8+ T cell). In some cases, the cell is a helper T cell (e.g., a CD4+ T cell). In some cases, the cell is a regulatory T cell (“Treg”). In some cases, the cell is a B cell. In some cases, the cell is a macrophage. In some cases, the cell is a dendritic cell. In some cases, the cell is a peripheral blood mononuclear cell. In some cases, the cell is a monocyte. In some cases, the cell is a natural killer (NK) cell. In some cases, the cell is a CD4+, FOXP3+ Treg cell. In some cases, the cell is a CD4+, FOXP3-Treg cell. The immune cell can be immunostimulatory or immunoinhibitory.

In some embodiments, the therapeutic cell may be a CAR T cell.

Suitable therapeutic cells also include stem cells, progenitor cells, as well as partially and fully differentiated cells. Suitable cells include neurons; liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); and a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cell is a stem cell. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

Cells of the present disclosure may be generated by any convenient method. Nucleic acids encoding one or more components of a subject circuit may be stably or transiently introduced into the subject immune cell, including where the subject nucleic acids are present only temporarily, maintained extrachromosomally, or integrated into the host genome. Introduction of the subject nucleic acids and/or genetic modification of the subject immune cell can be carried out in vivo, in vitro, or ex vivo.

As would be apparent, the cell may further comprise the target protein (i.e., a protein to which the fusion protein dimerizes and induces degradation of). In these embodiment the target protein, may be localized at the plasma membrane (either because it has a transmembrane sequence itself or it binds to a transmembrane protein) and comprises a target dimerization domain to which the first dimerization domain of (c)(i) binds. In these embodiments, binding of the fusion protein to the target protein via the first and target dimerization domains induces proteosome-mediated degradation of the target protein.

In some instances, the cell is obtained from an individual. For example, in some cases, the cell is a primary cell. As another example, the cell is a stem cell or progenitor cell obtained from an individual.

As one non-limiting example, in some cases, the cell is an immune cell obtained from an individual. As an example, the cell can be a T lymphocyte obtained from an individual. As another example, the cell is a cytotoxic cell (e.g., a cytotoxic T cell) obtained from an individual. As another example, the cell can be a helper T cell obtained from an individual. As another example, the cell can be a regulatory T cell obtained from an individual. As another example, the cell can be an NK cell obtained from an individual. As another example, the cell can be a macrophage obtained from an individual. As another example, the cell can be a dendritic cell obtained from an individual. As another example, the cell can be a B cell obtained from an individual. As another example, the cell can be a peripheral blood mononuclear cell obtained from an individual.

In some cases, the host cell is not an immune cell. In these embodiments, the host cell may be a somatic cell, e.g. a fibroblast, a hematopoietic cell, a neuron, a pancreatic cell, a muscle cell, a bone cell, a hepatocyte, a pancreatic cell, an epithelial cell, an endothelial cell, a cardiomyocyte, a T cell, a B cell, an osteocyte, or a stem cell, and the like.

As noted above, the rate at which the target protein is degraded may increase when the extracellular domain of the fusion protein and extracellular domain of the target transmembrane protein are bound to markers on the same cell. In these embodiments, the first dimerization domain and the target dimerization domain may bind to one another with a low affinity, as discussed above.

Protein circuits

The protein circuits of this disclosure provide a “NOT” gate, i.e., provide a way of inhibiting signaling within a cell in response to binding to a ligand on another cell (see e.g., Roybal et al Cell 2016 164: 770-9, Ebert et al Biochem Soc Trans. 2018 46: 391-401 and WO2005010198).

In these embodiments, the protein circuit may comprise i. a fusion protein as described above; and ii. a target protein comprising: an extracellular binding domain comprising a second binding moiety that is capable of specifically binding to a second cell surface marker, a transmembrane domain; and an intracellular domain that comprises a target dimerization domain to which the first dimerization domain of the fusion protein binds. In these embodiments, binding of the first binding moiety of the fusion protein and the second binding moiety of the target protein to cell surface markers that are on the same cell increases degradation of the target protein.

As described above, the fusion protein and the target protein both specifically bind to markers (i.e., proteins) that are on the surface of other cells. In some embodiments, the second cell surface marker (which is part of the target protein) may bind to a cancer marker, e.g., CD19, CD20, CD38, CD30, Her2/neu, ERBB2, CA125, MUC-1, prostate-specific membrane antigen (PSMA), CD44 surface adhesion molecule, mesothelin, carcinoembryonic antigen (CEA), epidermal growth factor receptor (EGFR), EGFRvIII, vascular endothelial growth factor receptor-2 (VEGFR2), high molecular weight-melanoma associated antigen (HMW-MAA), MAGE-A1, IL-13R-a2, GD2, and the like. Cancer-associated antigens also include, e.g., 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNTO888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-R α, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, tumor antigen CTAA16.88, VEGF-A, VEGFR-1, VEGFR2, or vimentin, for example.

In these embodiments, the first cell surface marker (which is part of the present fusion protein) may bind to cell surface protein on normal, non-cancerous cells, where the normal cells may also express the cancer-specific marker. This circuit results in degradation of the target protein in the cell, when the cell comes in contact with another cell that has the both the first and second markers on its surface. As such, the present circuit provides a way for therapeutic immune cells (e.g., chimeric antigen receptor (CAR) T cells) to discriminate between tumor and normal tissues in the treatment of cancer. Combinations of markers that could be used in the present circuits are described in, e.g., Dannenfelser et al (Cell Syst 2020 11: 215-228) and include, e.g., MLANA AND NOT MUC1 for the treatment of skin cutaneous melanoma, CA9 AND NOT VIPR1 for the treatment of renal clear cell carcinoma, GALNT1 (GD2) AND NOT AOC3, for the treatment of gliobastoma multiforme, PTPRN AND NOT LEPR for the treatment of testicular germ cell cancer, UPK2 AND NOT ROR 1 bladder carcinoma, ADAM12 AND NOT GPR133 for the treatment of breast carcinoma, MUC17 AND NOT PRR7 for the treatment of stomach adenocarcinoma, GRIN2D AND NOT LIFR for the treatment of colon adenocarcinoma, and CA9 AND NOT ABCA8 for the treatment of lung squamous cell carcinoma, where the fusion protein binds to the second protein listed in each pair (the “NOT” marker) and the target protein (which may part of a CAR) binds to the first protein listed in each pair. The following table lists several examples of pairs of antigens that can be combined with “NOT” gate logic, as well as an indication. In the following list the protein listed with an “L” may be the target protein in the following system, whereas the “H” protein may be targeted by an immune receptor such as a CAR.

| Combination | G | Indication |
|---|---|---|
| TNFRSF9:CACNG7 | LH | Brain Lower Grade Glioma |
| ROR2:TRPM1 | LH | Uveal Melanoma |
| ROR1:GPR143 | LH | Uveal Melanoma |
| KDR:TAS2R13 | LH | Acute Myeloid Leukemia |
| KDR:OR52H1 | LH | Acute Myeloid Leukemia |
| ROR1:MLANA | LH | Uveal Melanoma |
| EGFR:GPR143 | LH | Uveal Melanoma |
| EGFR:TRPM1 | LH | Uveal Melanoma |
| ROR1:TAS2R50 | LH | Acute Myeloid Leukemia |
| ERBB2:CACNG7 | LH | Glioblastoma Multiforme |
| TNFRSF9:CSPG5 | LH | Brain Lower Grade Glioma |
| ROR1:CACNG2 | LH | Pheochromocytoma and Paraganglioma |
| ROR2:TAS2R46 | LH | Acute Myeloid Leukemia |
| EPCAM:PCDHGC5 | LH | Glioblastoma Multiforme |
| TNFRSF9:NDRG4 | LH | Pheochromocytoma and Paraganglioma |
| CD80:ATP1A3 | LH | Pheochromocytoma and Paraganglioma |
| PROM1:ATP1A3 | LH | Pheochromocytoma and Paraganglioma |
| PROM1:KCNQ2 | LH | Pheochromocytoma and Paraganglioma |
| LEPR:L1CAM | LH | Uveal Melanoma |
| B4GALNT1:CD33 | LH | Acute Myeloid Leukemia |
| TNFRSF9:PTPRN | LH | Pheochromocytoma and Paraganglioma |
| B4GALNT1:LILRB4 | LH | Lymphoid Neoplasm Diffuse Large B-cell |
| | | Lymphoma |
| TNFRSF9:NTSR2 | LH | Glioblastoma Multiforme |
| CD80:SLC1A3 | LH | Brain Lower Grade Glioma |
| FOLH1:CD33 | LH | Acute Myeloid Leukemia |
| EPHA2:KCNQ2 | LH | Pheochromocytoma and Paraganglioma |
| MUC1:LYPD1 | LH | Glioblastoma Multiforme |
| ROR1:PRLHR | LH | Pheochromocytoma and Paraganglioma |
| FOLR1:CACNG2 | LH | Brain Lower Grade Glioma |
| MUC1:ZACN | LH | Acute Myeloid Leukemia |
| ROR1:RHAG | LH | Acute Myeloid Leukemia |
| EPHA2:TAS2R46 | LH | Acute Myeloid Leukemia |
| FOLH1:CACNG2 | LH | Brain Lower GradeGlioma |
| EPCAM:CNIH2 | LH | Glioblastoma Multiforme |
| EPHA2:OR52H1 | LH | Acute Myeloid Leukemia |
| MET:NETO1 | LH | Brain Lower Grade Glioma |
| EPHA2:GPR19 | LH | Glioblastoma Multiforme |
| CD80:CACNG7 | LH | Glioblastoma Multiforme |
| ERBB2:NETO1 | LH | Brain Lower Grade Glioma |
| ROR1:ULBP2 | LH | Head and Neck Squamous Cell Carcinoma |
| SCARA5:CA9 | LH | Kidney Renal Clear Cell Carcinoma |
| LIFR:CA9 | LH | Rectum Adenocarcinoma |
| ERBB2:L1CAM | LH | Pheochromocytoma and Paraganglioma |
| MUC1:PRLHR | LH | Pheochromocytoma and Paraganglioma |
| EPCAM:MLANA | LH | Skin Cutaneous Melanoma |
| EPHA2:CD79B | LH | Lymphoid Neoplasm Diffuse Large B-cell |
| | | Lymphoma |
| MUC1:STRA6 | LH | Testicular Germ Cell Tumors |
| CD274:TAS2R10 | LH | Acute Myeloid Leukemia |
| ERBB2:KISS1R | LH | Kidney Renal Clear Cell Carcinoma |
| AXL:ATP6AP2 | LH | Kidney Chromophobe |
| PROM1:SLCO1B1 | LH | Liver Hepatocellular Carcinoma |
| EPHA2:FAP | LH | Breast Invasive Carcinoma |
| TNFRSF9:TAS2R60 | LH | Acute Myeloid Leukemia |
| ERBB2:ATP8B3 | LH | Adrenocortical Carcinoma |
| ROR2:LINGO1 | LH | Brain Lower Grade Glioma |
| CA9:OR52B6 | LH | Acute Myeloid Leukemia |
| CD80:CNIH2 | LH | Brain Lower Grade Glioma |
| EGFR:KCNK15 | LH | Ovarian Serous Cystadenocarcinoma |
| MET:KCNA6 | LH | Brain Lower Grade Glioma |
| PROM1:SLC22A7 | LH | Liver Hepatocellular Carcinoma |
| ROR1:LY6G6D | LH | Rectum Adenocarcinoma |
| CDH1:IL13RA2 | LH | Pheochromocytoma and Paraganglioma |
| MUC1:SLC7A5 | LH | Skin Cutaneous Melanoma |
| B4GALNT1:LAT | LH | Thymoma |
| ROR1:KREMEN2 | LH | Head and Neck Squamous Cell Carcinoma |
| ROR1:PAQR9 | LH | Liver Hepatocellular Carcinoma |
| MUC1:ABCC11 | LH | Uveal Melanoma |
| KDR:PRR7 | LH | Ovarian Serous Cystadenocarcinoma |
| EPCAM:B4GALNT1 | LH | Glioblastoma Multiforme |
| CD80:APLP1 | LH | Pheochromocytoma and Paraganglioma |
| ROR1:SLC1A3 | LH | Glioblastoma Multiforme |
| RAMP3:MSLN | LH | Ovarian Serous Cystadenocarcinoma |
| PROM1:APLP1 | LH | Brain Lower Grade Glioma |
| FOLH1:KREMEN2 | LH | Cervical Squamous Cell Carcinoma and |
| | | Endocervical Adenocarcinoma |
| EPHA2:P2RX5 | LH | Lymphoid Neoplasm Diffuse Large B-cell |
| | | Lymphoma |
| ROR1:GRIN2D | LH | Colon Adenocarcinoma |
| MET:OR51E2 | LH | Prostate Adenocarcinoma |
| ROR2:LILRB4 | LH | Lymphoid Neoplasm Diffuse Large B-cell |
| | | Lymphoma |
| EPCAM:TMPRSS11D | LH | Head and Neck Squamous Cell Carcinoma |
| MET:SPN | LH | Acute Myeloid Leukemia |
| AOC3:AXL | LH | Glioblastoma Multiforme |
| ROR1:RGR | LH | Brain Lower Grade Glioma |
| KDR:MLC1 | LH | Glioblastoma Multiforme |
| CD80:TYRO3 | LH | Uveal Melanoma |
| EPCAM:BSG | LH | Uveal Melanoma |
| GPR133:BSG | LH | Kidney Chromophobe |
| EPHA2:OR2B6 | LH | Breast Invasive Carcinoma |
| ROR1:GPR35 | LH | Colon Adenocarcinoma |
| FOLH1:LAT | LH | Thymoma |
| EPCAM:PLXNA1 | LH | Skin Cutaneous Melanoma |
| FAP:CLEC12B | LH | Acute Myeloid Leukemia |
| MUC1:B4GALNT1 | LH | Pheochromocytoma and Paraganglioma |
| SFRP1:CEACAM5 | LH | Colon Adenocarcinoma |
| FOLR1:ATP1A3 | LH | Brain Lower Grade Glioma |
| MUC1:TFR2 | LH | Liver Hepatocellular Carcinoma |
| KDR:OR2B6 | LH | Cervical Squamous Cell Carcinoma and |
| | | Endocervical Adenocarcinoma |
| MUC1:SLC22A9 | LH | Liver Hepatocellular Carcinoma |
| FOLH1:PTPRCAP | LH | Lymphoid Neoplasm Diffuse Large B-cell |
| | | Lymphoma |
| EGFR:CD72 | LH | Lymphoid Neoplasm Diffuse Large B-cell |
| | | Lymphoma |
| EPCAM:TAS2R30 | LH | Acute Myeloid Leukemia |
| MUC1:CD83 | LH | Lymphoid Neoplasm Diffuse Large B-cell |
| | | Lymphoma |
| EGFR:SLC7A5 | LH | Skin Cutaneous Melanoma |
| ROR1:OR2B6 | LH | Testicular Germ Cell Tumors |
| MET:MSLN | LH | Ovarian Serous Cystadenocarcinoma |

Several other pairs of NOT gated antigens are known (see, e.g., the antigen.princeton website) that can use used herein.

### Nucleic Acids

Given that the genetic code is known, a sequence that encodes the fusion protein can be readily determined. In some embodiments, the coding sequence may be codon optimized for expression in mammalian (e.g., human or mouse) cells, strategies for which are well known (see, e.g., Mauro et al., Trends Mol. Med. 2014 20: 604-613 and Bell et al Human Gene Therapy Methods 27: 6). As would be understood, the coding sequence may be operably linked to a promoter, which may be inducible, tissue-specific, or constitutive. In some embodiments, the promoter may be activated by an engineered transcription factor that is heterologous to the cell, e.g., a Gal4-, LexA-, Tet-, Lac-, dCas9-, zinc-finger- and TALE-based transcription factors.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF”' state during specific stages of embryonic development or during specific stages of a biological process, e.g., hair follicle cycle in mice).

For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein ((TA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a CD8 cell-specific promoter, a CD4 cell-specific promoter, a neutrophil-specific promoter, or an NK-specific promoter. For example, a CD4 gene promoter can be used; see, e.g., Salmon et al. (1993) Proc. Natl. Acad. Sci. USA 90: 7739; and Marodon et al. (2003) Blood 101: 3416. As another example, a CD8 gene promoter can be used. NK cell-specific expression can be achieved by use of an Nerl (p46) promoter; see, e.g., Eckelhart et al. (2011) Blood 117: 1565.

In some cases, the promoter is a cardiomyocyte-specific promoter. In some cases, the promoter is a smooth muscle cell-specific promoter. In some cases, the promoter is a neuron-specific promoter. In some cases, the promoter is an adipocyte-specific promoter. Other cell type-specific promoters are known in the art and are suitable for use herein.

Suitable expression vectors include, but are not limited to, viral vectors (e.g. viral vectors based on vaccinia virus; poliovirus; adenovirus (see, e.g., Li et al., Invest Opthalmol Vis Sci 35: 2543 2549, 1994; Borras et al., Gene Ther 6: 515 524, 1999; Li and Davidson, PNAS 92: 7700 7704, 1995; Sakamoto et al., Hum Gene Ther 5: 1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9: 81 86, 1998, Flannery et al., PNAS 94: 6916 6921, 1997; Bennett et al., Invest Opthalmol Vis Sci 38: 2857 2863, 1997; Jomary et al., Gene Ther 4: 683 690, 1997, Rolling et al., Hum Gene Ther 10: 641 648, 1999; Ali et al., Hum Mol Genet 5: 591 594, 1996; Srivastava in WO 93/09239, Samulski et al., J. Vir. (1989) 63: 3822-3828; Mendelson et al., Virol. (1988) 166: 154-165; and Flotte et al., PNAS (1993) 90: 10613-10617); SV40; herpes simplex virus; human immunodeficiency virus (see, e.g., Miyoshi et al., PNAS 94: 10319 23, 1997; Takahashi et al., J Virol 73: 7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, the vector is a lentivirus vector. Also suitable are transposon-mediated vectors, such as piggyback and sleeping beauty vectors.

### Methods

Also provided herein is method for inducing degradation of a target protein in the cell above. In these embodiments, the method may comprise introducing a first cell to a second cell, wherein: (a) the first cell comprises: i. a fusion protein as described above; and

ii. a target protein, wherein the target protein comprises: an extracellular binding domain comprising a second binding moiety that is capable of specifically binding to a cell surface marker; a transmembrane domain; and an intracellular domain that comprises a target dimerization domain to which the first dimerization domain of the fusion protein binds (e.g., with a low affinity); and (b) the second cell comprises, on its surface, the first and second cell surface markers. In this method, binding of the first cell to the second brings the fusion protein and the target protein into proximity, thereby causing them to dimerize together to result in an increase in the degradation of the target protein. As noted above, the first cell may be an immune cell, the target protein is a chimeric antigen receptor (or TCR), and the second cell may be a non-cancerous cell. The introducing can be done in vitro (using isolated cells), ex vivo (using cells that have been taken from a person) or in vivo. In the latter case, the introducing may be done by administering the first cell to a subject, e.g., by injection.

In some embodiments, the cell may be used in a method of treatment that comprises administering the cell to a patient in need thereof.

In some embodiments, the patient may have cancer, e.g., breast cancer, B cell lymphoma, pancreatic cancer, Hodgkin lymphoma cell, ovarian cancer cell, prostate cancer, mesothelioma, lung cancer (e.g., a small cell lung cancer), non-Hodgkin B-cell lymphoma (B-NHL) cell, ovarian cancer, a prostate cancer, melanoma cell, a chronic lymphocytic leukemia cell, acute lymphocytic leukemia cell, a neuroblastoma, a glioma, a glioblastoma, a medulloblastoma, a colorectal cancer, etc. In these embodiments, the therapeutic cell may be a CAR T cell that comprises a CAR that recognizes an antigen expressed by the cancer cells.

In some embodiments, the patient may have an inflammatory condition or autoimmune disease. In these embodiments, the cell may be T-helper cell or a Tregs for use in an immunomodulatory method. Immunomodulatory methods include, e.g., enhancing an immune response in a mammalian subject toward a pathogen; enhancing an immune response in a subject who is immunocompromised; reducing an inflammatory response; reducing an immune response in a mammalian subject to an autoantigen, e.g., to treat an autoimmune disease; and reducing an immune response in a mammalian subject to a transplanted organ or tissue, to reduce organ or tissue rejection.

In some embodiments, the patient is in need of a stem cell transplantation.

### EXAMPLES

The following examples are put forth so as to provide those of ordinary skill in the art with a complete disclosure and description of how to make and use the present invention, and are not intended to limit the scope of what the inventors regard as their invention.

### Example 1

### Cis-Ubiquitination can be Prevented by Substituting the Lysines in a STUD

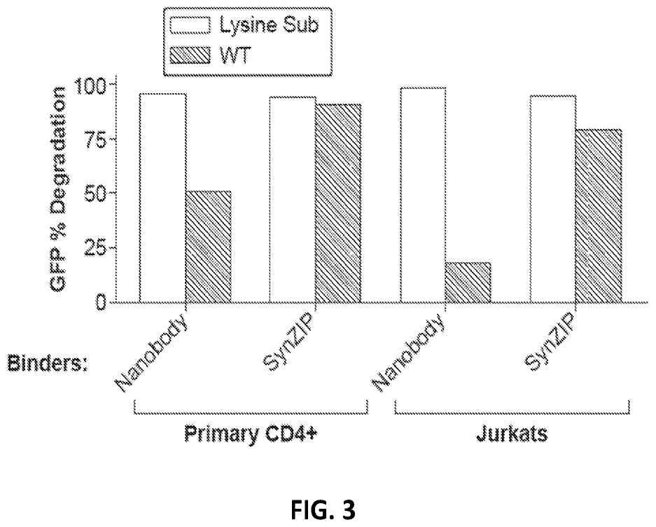

This protein degradation tool has the potential to ubiquitinate target lysines on both the target of interest (trans-ubiquitination), as well as on the tool itself (cis-ubiquitination). cis-ubiquitination may limit the effectiveness of the STUD by degrading the STUD before it has the chance to interact with its target. To solve this problem, the lysines on the protein targeting domain of the STUD were mutated to arginines (K->R), thus preventing cis-ubiquitination². An assay was developed to test the functionality of a STUD by measuring degradation of a cytosolic GFP. The GFP was targeted for degradation using either a GFP nanobody or a SynZIP17 that was fused to the GFP. The target GFP was transduced into either Jurkat cells or primary human T cells using lentivirus and the STUD was introduced via a second lentivirus. It was observed that the lysine substitution significantly improved the activity of the GFP nanobody STUD, whereas the mutation only moderately improved the activity of the SynZIP STUD. These results are shown in FIG. 3. This trend was consistent between primary human CD4+ T cells and Jurkats. Given these results, it should be possible to use the number of lysines on the STUD as a strategy for tuning the activity of the STUD, where more mutated lysines increases the activity of the STUD.

### Example 2

### STUD-Induced Degradation is Mediated Via the Proteasome

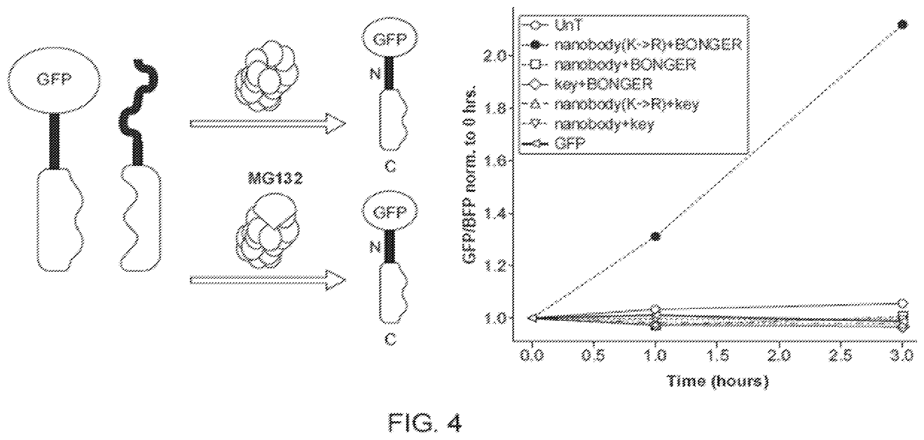

The mechanism of how the STUD reduces GFP was explored. Primary human CD4+ T cells expressing the GFP nanobody STUD were fed with the MG132 proteasome inhibitor and the change in fluorescence was measured over time. These results are shown in FIG. 4. Cells expressing a functional STUD should display an increase in fluorescence over time as the proteasome inhibitor took effect. After three hours of exposure to the drug, it was observed that only the cells expressing the functional nanobody STUD (nanobody(K->R)+Bonger) displayed an increase in GFP fluorescence. This indicates that the observed reduction in GFP is mediated by degradation via the proteasome rather than a mechanism associated with the protein-protein interaction alone.

### Example 3

### STUD Activity can be Optimized Using a Linker

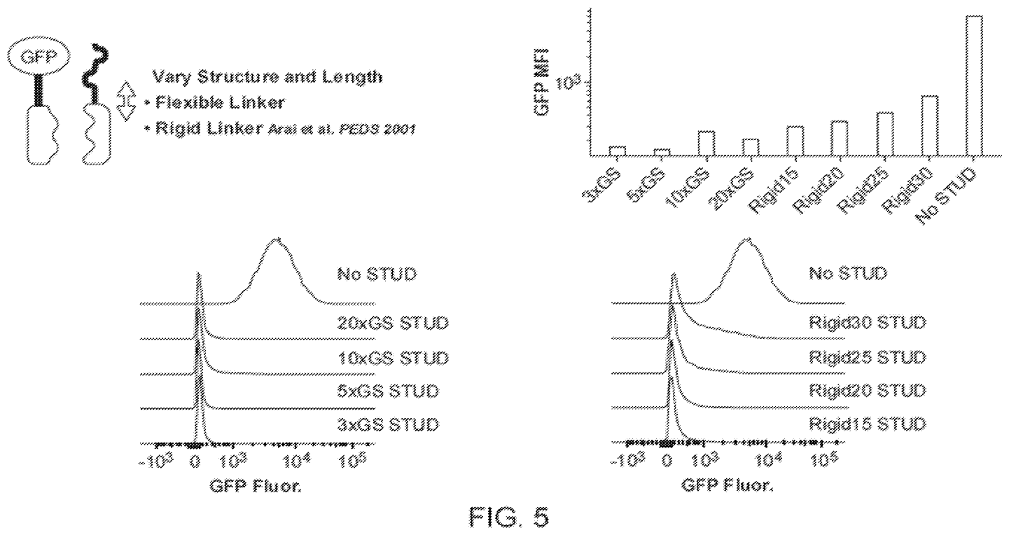

The STUD was optimized by screening multiple lengths of two different classes of linkers. In these constructs, the linker was added between a SynZIP protein binding domain and the Bonger degron. It was hypothesized that a flexible Gly-Ser linker may facilitate target degradation by increasing the accessibility of the E3 ligase to reach target lysine residues on the surface of the target protein, whereas a rigid helical linker may increase the distance between the E3 ligase and target lysines and reduce degradation. These experiments used the SynZIP STUD that targets cytosolic GFP-SZ17 as described above. Four lengths of linker for both the flexible and rigid linker. The flexible linker generally performed better than the rigid linker, with little variation in degradation efficiency observed within the different flexible linker lengths (FIG. 5). However among the flexible linkers the 5xGS performed the best. This STUD (with the SynZIP(K->R), optimized linker and C-terminal RRRG, or SynZIP18(K->R)-5xGS-RRRG) is referred to as the “soluble stud” and used in the following experiments.

### Example 4

### Transcription Factors can be Targeted

Lysine substitution and linker length/type optimization served as a framework for optimizing future STUD iterations that use other protein targeting domains and/or degradation domains, e.g., degrons. Depending on the application, different synthetic protein targeting domains may be more suitable, and it is also possible to utilize endogenous protein targeting domains that bind to or interact with an endogenous protein without the need for modification of the endogenous protein. Furthermore, different degrons may be utilized to vary the conditions under which the STUD is active, or confine the activity of the STUD to different compartments of the cell where the degron is active.

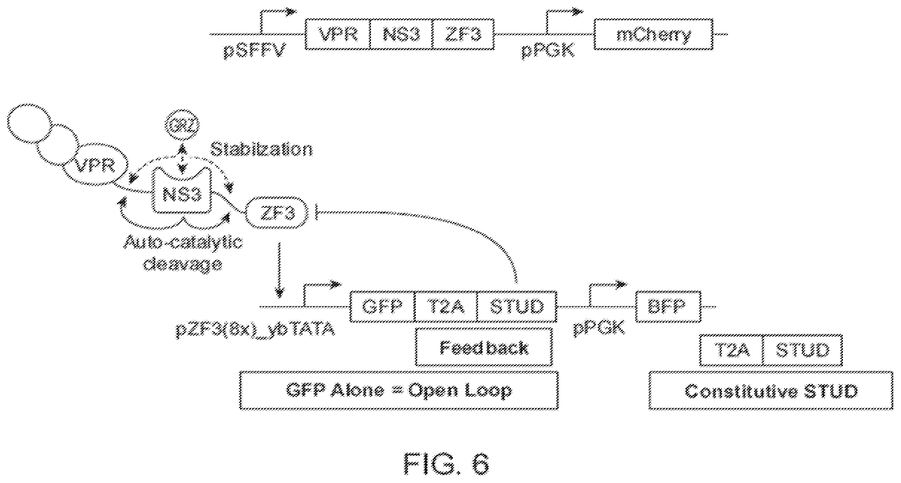

A transcription factor was targeted for degradation using the soluble STUD described above. Modulating a transcription factor allows one to affect the output of a functional protein. These experiments were done using a previously developed grazoprevir (GRZ) drug-inducible zinc-finger transcription factor system (VPR-NS3-ZF3). To induce degradation of this transcription factor SynZIP17 to the C-terminus of this protein. Degradation of the TF was measured by observing changes in GFP reporter output driven by the pZF3(8x)ybTATA promoter. Two different methods were used for STUD expression: constitutive STUD expression, or inducible STUD expression, which should drive negative feedback in the system (FIG. 6).

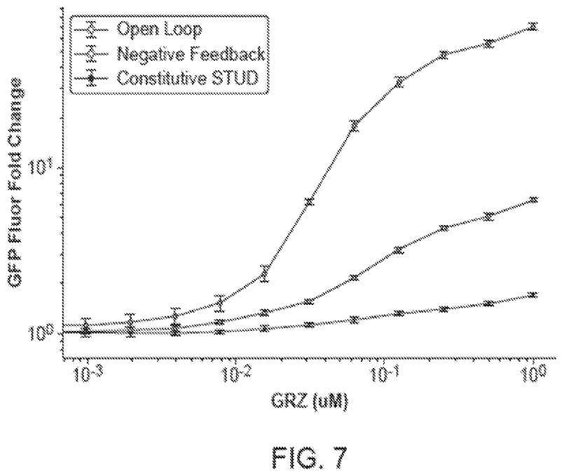

The dose responses of the three circuit variants were compared to assess the functionality of the STUD. It was found that constitutive expression of the STUD abolished nearly all output from the pZF3, whereas feedback expression of the STUD generated an intermediate dose response (FIG. 7). This demonstrates that the soluble STUD can not only degrade functional proteins in the cell, but also be used as a powerful tool for building genetic circuits.

### Example 5

### Transmembrane Proteins can be Targeted

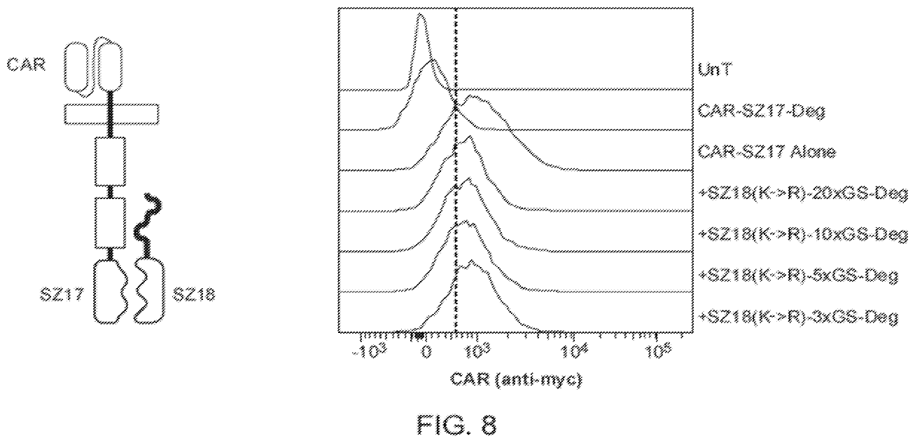

Next, the soluble STUD was used to target a membrane protein for degradation. The ability of the STUD to degrade a CAR in Jurkat cells was tested by generating a CAR construct with SynZIP17 fused to its C-terminus. However, while these STUDs worked to some extent, none of them were able to completely knockdown CAR expression (see FIG. 8).

It was found that the Bonger degron, when directly fused to the CAR, was able to reduce CAR expression by over 90%. This result suggested that the soluble STUD was not working due to insufficient interaction with the CAR, rather than a defect with the ability of the degron to target membrane proteins for degradation.

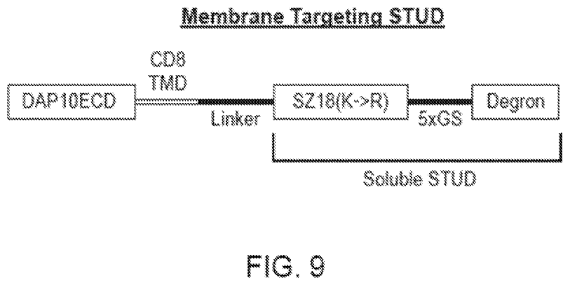

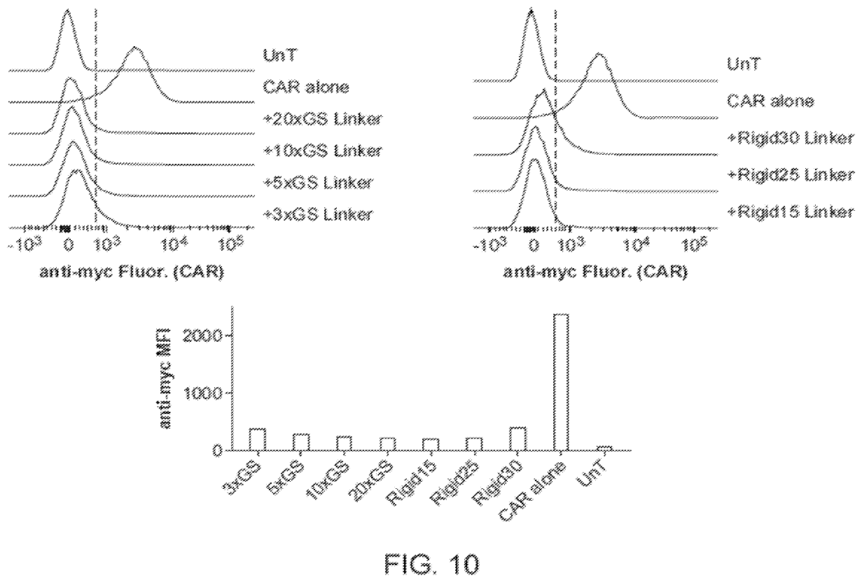

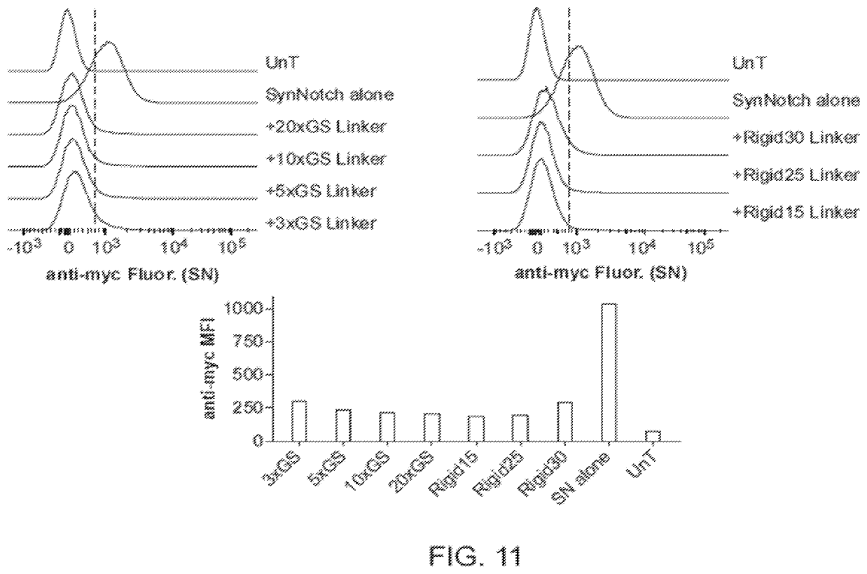

To increase the likelihood of interaction between the STUD and the CAR, a new STUD construct that was itself localized to the membrane using the DAP10 signal sequence was generated (FIG. 9). A library of linkers between the CD8 transmembrane domain and the soluble STUD was also tested. The ability of these new membrane targeting STUDs were tested for their ability to degrade both a CAR and a SynNotch in primary human CD4+ T cells. It was found that the best results were provided using a rigid linker between the CD8 TMD and STUD. All linkers were effective, but use of the Rigid15 linker resulted in over 95% knock-down of CAR expression as measured by surface staining for CAR expression (FIG. 10). This result was replicated for a membrane targeting STUD targeting a SynNotch for degradation (FIG. 11).

### Materials and Methods

Cytosolic STUD for targeting GFP: Cytosolic STUDs were introduced by lentiviral transduction of two plasmids. The first encodes a green fluorescent protein (GFP) which will be a target for degradation alongside a BFP as a co-transduction marker. The second encodes the STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of GFP fluorescence.

Using proteasome inhibitor to explore cytosolic GFP mechanism: To ascertain the mechanism by which the STUD degrades cytosolic GFP, we incubated cells with 5 M of the proteasome inhibitor MG132 for 1 and 3 hours. Cells were then washed with PBS and analyzed by flow cytometry. Using the same 2-plasmid system as described above, we measured changes in GFP fluorescence relative to controls.

Membrane targeting STUD: Membrane targeting cells were introduced by lentiviral transduction of two plasmids. The first encodes a chimeric antigen receptor (CAR) or synthetic Notch (SynNotch) protein which will be a target for degradation alongside a BFP as a co-transduction marker. The second encodes the membrane localized STUD protein, or non-functional controls, alongside an mCherry fluorescent protein as a co-transduction marker. Cells were then analyzed by flow cytometry. Cells were gated on expression of co-transduction fluorescent proteins (BFP/mCherry) and STUD efficacy was measured by knockdown of CAR/SynNotch. CAR and SynNotch expression was measured by antibody staining for a peptide tag fused to the extracellular domain of the CAR/SynNotch.

### REFERENCES

1. Bonger, K. M., Chen, L.-C., Liu, C. W. & Wandless, T. J. Small-molecule displacement of a cryptic degron causes conditional protein degradation. Nat. Chem. Biol. 7, 531-537 (2011).

2. Daniel, K. et al. Conditional control of fluorescent protein degradation by an auxin-dependent nanobody. Nat. Commun. 9, 3297 (2018).

3. Arai, R., Ueda, H., Kitayama, A., Kamiya, N. & Nagamune, T. Design of the linkers which effectively separate domains of a bifunctional fusion protein. Protein Eng. 14, 529-532 (2001).

4. Wu, C. Y., Roybal, K. T., Puchner, E. M. & Onuffer, J. Remote control of therapeutic T cells through a small molecule-gated chimeric receptor. Science (2015).

What is claimed is:

1. A fusion protein comprising:
(a) an extracellular domain comprising a first binding moiety that is capable of specifically binding to a first cell surface marker;
(b) a transmembrane domain; and
(c) an intracellular domain comprising:
i. a first dimerization domain that specifically binds to a corresponding target dimerization domain in a target protein; and
ii. a degradation domain, wherein the degradation domain is a degron or E3 ligase-recruiting domain.

2. The fusion protein of claim 1, wherein the first dimerization domain binds to the target dimerization with a low affinity.

3. The fusion protein of claim 1 or 2, wherein the first dimerization domain and target dimerization domains are synthetic leucine zipper domains.

4. The fusion protein of claim 3, wherein at least one of the synthetic leucine zipper domains has up to seven α-helical turns.

5. The fusion protein of any of claims 1-4, wherein the degradation domain is a degron.

6. The fusion protein of any of claims 1-4, wherein the degradation domain is an E3 ligase-recruiting domain.

7. The fusion protein of any of claims 1-6, wherein the extracellular domain of the fusion protein comprises a scFv, a nanobody or a ligand for a cell-surface receptor.

8. A cell comprising a fusion protein of any of claims 1-7, or a nucleic acid containing the same.

9. The cell of claim 8, wherein the cell further comprises:
the target protein,
wherein the target protein is localized at the plasma membrane and comprises a target dimerization domain to which the first dimerization domain of (c)(i) binds, and
wherein binding of the fusion protein to the target protein via the first and target dimerization domains induces proteosome-mediated degradation of the target protein.

10. The cell of claim 9, wherein the target protein is a transmembrane protein.

11. The cell of claim 10, wherein the target protein comprises:
i. an extracellular binding domain comprising a second binding moiety that is capable of specifically binding to a second cell surface marker;
ii. a transmembrane domain; and
iii. an intracellular domain that comprises the target dimerization domain.

12. The cell of claim 11, wherein the intracellular domain further comprises an effector region that is activated by binding of the extracellular binding domain to a target via the first binding region.

13. The cell of claim 9, wherein the target protein is associated with a transmembrane protein.

14. The cell of any of claim 9, 10, 12 or 13, wherein the target protein is a chimeric antigen receptor.

15. The cell of any of claims 9-14, wherein the first dimerization domain and the target dimerization domain are synthetic leucine zipper dimerization domains.

16. The cell of any of claims 9-15, wherein the first dimerization domain and the target dimerization domain bind to one another with a low affinity, and the rate at which the target protein is degraded increases when the extracellular domain of the fusion protein and the extracellular domain target transmembrane protein are both bound markers to the same cell.

17. The cell of any of claims 9-16, wherein said cell is an immune cell.

18. The cell of claim 17, wherein the cell is a T cell, macrophage or natural killer (NK) cell.

19. A protein circuit comprising
i. a fusion protein of any of claims 1-7; and
ii. a target protein comprising:
(i) an extracellular binding domain comprising a second binding moiety that is capable of specifically binding to a second cell surface marker;
(ii) a transmembrane domain; and
(iii) an intracellular domain that comprises a target dimerization domain to which the first dimerization domain of the fusion protein binds;
wherein binding of the first binding moiety of the fusion protein and the second binding moiety of the target protein to cell surface markers that are on the same cell increases degradation of the target protein.

20. The protein circuit of claim 19, wherein the target dimerization domain and the first dimerization domain bind to one another with a low affinity.

21. A method for inducing degradation of a target protein, comprising introducing a first cell to a second cell, wherein:
(a) the first cell comprises:
i. a fusion protein of claim 1; and
ii. a target protein, wherein the target protein comprises:
(i) an extracellular binding domain comprising a second binding moiety that is capable of specifically binding to a second cell surface marker;
(ii) a transmembrane domain; and
(iii) an intracellular domain that comprises a target dimerization domain to which the first dimerization domain of the fusion protein binds with a low affinity; and
(b) the second cell comprises, on its surface, the first and second cell surface markers;
thereby inducing degradation of the target protein.

22. The method of claim 21, wherein the first cell is an immune cell, the target protein is a chimeric antigen receptor, and the second cell is a non-cancerous cell.

23. The method of claim 21 or 22, wherein the first dimerization domain and the target dimerization domain are synthetic leucine zipper domains.

24. The method of any of claims 21-23, wherein the introducing is done by administering the first cell to a subject.
